(19) United States
(12) Patent Application Publication
(10) Pub. No.: US 2023/0128299 A1
(43) Pub. Date: Apr. 27, 2023

(54) CLOUD SERVER, COMPUTER READABLE MEDIUM, AND CLOUD SYSTEM

(71) Applicant: Konica Minolta, Inc., Tokyo (JP)

(72) Inventor: Takafumi Nanjo, Tokyo (JP)

(73) Assignee: Konica Minolta, Inc., Tokyo (JP)

(21) Appl. No.: 17/973,779

(22) Filed: Oct. 26, 2022

(30) Foreign Application Priority Data
Oct. 26, 2021 (JP) 2021-174313

(51) Int. Cl. G16H 15/00; G16H 30/20; G16H 40/67

(52) U.S. Cl. CPC G16H 15/00; G16H 30/20; G16H 40/67

(57) ABSTRACT

A cloud server that manages a medical image and is connected to a local server through a network includes: a hardware processor that: acquires a first interpretation report on a first medical image from the local server through an internet connection, and acquires a second medical image from the local server through the internet connection and creates a second interpretation report on the second medical image; a first storage that stores the first interpretation report; and a second storage that stores the second interpretation report.

### CROSS-REFERENCE TO RELATED APPLICATIONS

The entire disclosure of Japanese Patent Application No. 2021-174313 filed on Oct. 26, 2021 is incorporated herein by reference.

### BACKGROUND

### Technical Field

The present invention relates to a cloud server, a computer-readable medium, and a cloud system.

### Description of Related Art

Medical images captured in medical facilities, such as clinics and hospitals, are usually stored in a medical image management system called a picture archiving and communication system (PACS). A doctor interprets the medical image and creates an interpretation report as a result.

In recent years, a system has been used that uploads medical information, such as medical images captured in hospitals and diagnostic reports created by diagnosticians, to medical information storages on the cloud to publish medical information on the cloud to relevant hospitals and companies (see JP 2014-44495 A). In the technique described in JP 2014-44495 A, the disclosure range is determined based on the reference authority set in the medical information.

However, in the known technique described above, reports created in medical facilities such as hospitals are uploaded to the cloud, but the data in the server in the medical facility is positioned as a master and the data in the cloud server is positioned as a backup.

In recent years, in order to improve diagnostic performance, multiple interpretations of medical images to be diagnosed have been recommended. Multiple interpretations mean that a plurality of doctors interprets the same examination (medical image). If there are a plurality of doctors (interpretation doctors) in the medical facility, multiple interpretations using a server in the medical facility (on-premise server) are possible. However, in many small clinics, there is only one doctor. If such a clinic is not included in a group of hospitals that can cooperate with each other through VPN connections, it is necessary to perform multiple interpretations using a server in the clinic, and it is not realistic for a plurality of doctors to create interpretation reports for the same medical image. That is, even in small clinics that are not connected to other hospitals through VPN connections, it is required that doctors belonging to other hospitals can create interpretation reports for the same medical image. In the case of electronic medical charts, a plurality of doctors does not create medical charts for the same subject. For this reason, this issues especially in interpretation reports.

In recent years, work style reforms for doctors have been recommended, and it is desired that doctors can not only create interpretation reports on a local server in the hospital, but also create interpretation reports at home or on the go.

### SUMMARY

One or more embodiments of the present invention provide a practical and technological improvement over conventional could server, computer-readable medium, and cloud system. For example, one or more embodiments enable the creation of an interpretation report on a cloud server.

According to an aspect of the present invention, a cloud server that manages a medical image and is connected to a local server through a network comprises: a hardware processor that acquires a first interpretation report on a first medical image from the local server through the Internet or an internet connection, acquires a second medical image from the local server through the Internet, and creates a second interpretation report on the second medical image; a first storage that stores the first interpretation report; and a second storage that stores the second interpretation report.

According to another aspect of the present invention, a non-transitory computer-readable medium stores a program to be executed by a computer of a cloud server included in a cloud system in which a local server and the cloud server for managing a medical image are connected to each other through a network, wherein the program causes the computer to perform: acquiring a first interpretation report on a first medical image from the local server through the Internet or an internet connection; storing the first interpretation report; acquiring a second medical image from the local server through the Internet; creating a second interpretation report on the second medical image; and storing the second interpretation report.

According to another aspect of the present invention, a non-transitory computer-readable medium stores a program to be executed by a computer of a local server included in a cloud system in which a local server and the cloud server for managing a medical image are connected to each other through a network, wherein the program causes the computer to perform: transmitting a first interpretation report on a first medical image and a second medical image to the cloud server through the Internet or an internet connection; acquiring a second interpretation report on the second medical image created in the cloud server; and storing the second interpretation report.

According to another aspect of the present invention, a cloud system in which a local server and a cloud server for managing a medical image are connected to each other through a network comprises: a hardware processor that acquires a first interpretation report on a first medical image from the local server through the Internet or an internet connection, acquires a second medical image from the local server through the Internet, and creates a second interpretation report on the second medical image; a first storage that stores the first interpretation report; and a second storage that stores the second interpretation report.

According to another aspect of the present invention, a cloud system includes a plurality of local servers including a first local server and a second local server and the cloud server described above that is connected to the plurality of local servers through a network, wherein the cloud server is accessible from the first local server and the second local server and the first local server and the second local server are inaccessible to each other.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a system configuration diagram of a cloud system according to a first embodiment of the present invention;

FIG. 2 is a block diagram showing the functional configuration of a cloud server;

FIG. 3 is a diagram showing a data configuration example of a user table;

FIG. 4 is a diagram showing a data configuration example of a group table;

FIG. 5 is a diagram showing a data configuration example of an examination management table;

FIG. 6 is a diagram showing a data configuration example of a report management table;

FIG. 7 is a block diagram showing the functional configuration of a local server;

FIG. 8 is a block diagram showing the functional configuration of a client terminal;

FIG. 9 is a ladder chart showing a first interpretation process;

FIG. 10 is a ladder chart showing a second interpretation process;

FIG. 11 is an example of an interpretation screen displayed on a client terminal;

FIG. 12 is a ladder chart showing a report display process;

FIG. 13 is an example of a report reference screen displayed on a client terminal;

FIGS. 14A and 14B are examples of a report reference screen when a client terminal has two screen monitors;

FIG. 15 is an example of a report reference screen displayed on a client terminal in a second embodiment; and

FIG. 16 is a ladder chart showing a second report display process in a third embodiment.

### DETAILED DESCRIPTION OF THE EMBODIMENTS

In one or more embodiments of the present invention, examples of a local server include a server that can be accessed merely by local connection without the Internet.

Examples of the local server include a physical server that is shared in one or more facilities and can be accessed merely by local connection.

In one or more embodiments of the present invention, a local server is accessed within a local network, and a first interpretation report is created within the local network for medical images stored in the local server.

In one or more embodiments of the present invention, examples of a cloud server include a server constructed under a cloud environment accessible through an Internet connection.

For example, in an environment in which a plurality of local servers including a first local server and a second local server are indirectly network-connected through a cloud server, the first local server and the second local server are not included in the cloud server because the first local server and the second local server are indirectly connected to each other through the cloud server.

Examples of the cloud server include a virtual server used in the range of the storage capacity given from one or more facilities through the Internet, which is a large-capacity server provided by a cloud service provider.

In one or more embodiments of the present invention, a user accesses a cloud server through the Internet, and a second interpretation report is created through the Internet for medical images stored in the cloud server.

Hereinafter, a cloud system according to one or more embodiments of the present invention will be described with reference to the accompanying diagrams. However, the scope of the present invention is not limited to the illustrated examples.

### First Embodiment

<Configuration of Cloud System>

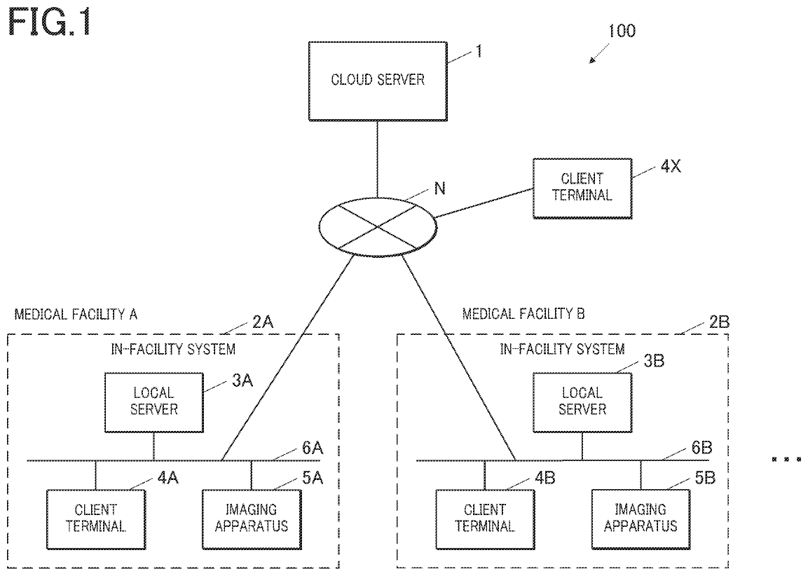

FIG. 1 shows the system configuration of a cloud system 100 according to a first embodiment.

As shown in FIG. 1, the cloud system 100 is configured to include a cloud server 1 installed in a cloud environment, an in-facility system 2A provided in a medical facility A, an in-facility system 2B provided in a medical facility B, a client terminal 4X, and the like. The cloud server 1 is connected to the in-facility system 2A, the in-facility system 2B, the client terminal 4X, and the like through a communication network N, such as the Internet, so that data communication therebetween is possible.

The cloud server 1 stores and manages image data of medical images created in each medical facility. The cloud server 1 receives requests for interpretation of medical images from the in-facility systems 2A, 2B, . . . , and stores and manages interpretation reports.

The in-facility system 2A includes a local server 3A, a client terminal 4A, and an imaging apparatus 5A, and these are connected to each other by an in-facility network 6A, such as a local area network (LAN), so that data communication therebetween is possible. The local server 3A and the client terminal 4A are connected to the cloud server 1 through the communication network N so that data communication therebetween is possible.

The in-facility system 2B includes a local server 3B, a client terminal 4B, and an imaging apparatus 5B, and these are connected to each other by an in-facility network 6B, such as a LAN, so that data communication therebetween is possible. The local server 3B and the client terminal 4B are connected to the cloud server 1 through the communication network N so that data communication therebetween is possible.

The client terminal 4X can connect to the cloud server 1 through the communication network N to perform data communication regardless of whether the client terminal 4X is located inside or outside the medical facility.

In the cloud system 100, the cloud server 1 is connected to a plurality of local servers 3A, 3B, . . . , through the communication network N.

The cloud server 1 can be accessed from at least the local server 3A (first local server) and the local server 3B (second local server) among the plurality of local servers 3A, 3B, . . . .

On the other hand, local servers between different in-facility systems, such as the local server 3A (first local server) and the local server 3B (second local server), are inaccessible to each other.

The number of in-facility systems such as the in-facility systems 2A and 2B forming the cloud system 100 and the number of client terminals inside or outside each in-facility system are not particularly limited.

<Configuration of Cloud Server>

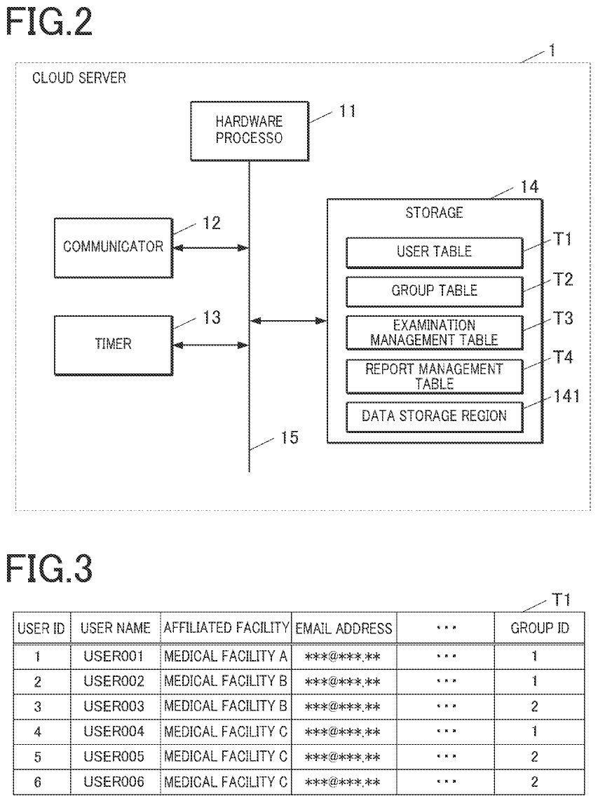

FIG. 2 shows the functional configuration of the cloud server 1.

As shown in FIG. 2, the cloud server 1 is configured to include a hardware processor 11, a communicator 12, a timer 13, a storage 14, and the like, and these are connected to each other by a bus 15.

The hardware processor 11 includes a central processing unit (CPU), a random access memory (RAM), and the like, and performs overall control of the processing operation of each unit of the cloud server 1. Specifically, the CPU reads various processing programs stored in the storage 14, loads the programs to the RAM, and performs various processes in cooperation with the programs.

The communicator 12 includes a network interface and the like, and transmits and receives data to and from an external apparatus connected through the communication network N.

The timer 13 has a real time clock (RTC), and measures the current date and time by using the real time clock and outputs the current date and time to the hardware processor 11.

The storage 14 includes a hard disk drive (HDD), a solid state drive (SSD), and the like, and stores various processing programs and parameters, files, and the like necessary for executing the programs. For example, a web server program for realizing a function as a web server that communicates with a web browser installed in an external apparatus through an HTTP protocol to provide various web screens to the web browser or an application program that operates on a web server and provides an image viewer function, an interpretation report creation function, an interpretation report display function, and the like to the user of an external apparatus through a web browser is stored in the storage 14.

The storage 14 stores a user table T1, a group table T2, an examination management table T3, a report management table T4, and the like. The storage 14 has a data storage region 141.

The user table T1 is a table for managing user information regarding each user who uses the cloud system 100. FIG. 3 shows an example of the data configuration of the user table T1. In the user table T1, a user ID, a user name, an affiliated facility, an email address, a group ID, and the like are stored for each user so as to be associated with each other. The user ID is identification information of the user. The user name is the name of the user. The affiliated facility is a medical facility to which the user belongs. The email address is the user's email address. The group ID is the group ID of a group to which the user belongs.

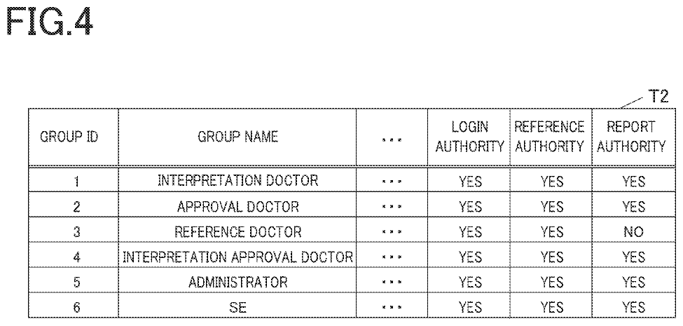

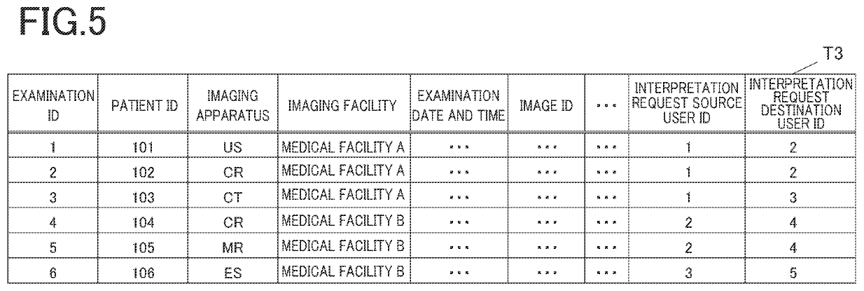

The group table T2 is a table that defines the authority for the cloud server 1 for each group to which the user belongs. FIG. 4 shows an example of the data configuration of the group table T2. In the group table T2, a group ID, a group name, login authority, reference authority, report authority, and the like are stored for each group so as to be associated with each other. The group ID is identification information of the group. The group name is the name of the group (interpretation doctors, approval doctors, reference doctors, and the like). The login authority is the authority to log in to the system provided by the cloud server 1. The reference authority is the authority to refer to medical images or interpretation reports managed by the cloud server 1. The report authority is the authority to create an interpretation report on the cloud server 1.

The examination management table T3 is a table for managing examinations relevant to medical images stored in the data storage region 141. FIG. 5 shows an example of the data configuration of the examination management table T3. In the examination management table T3, an examination ID, a patient ID, an imaging apparatus, an imaging facility, an examination date and time, an image ID, an interpretation request source user ID, an interpretation request destination user ID, and the like are stored for each examination so as to be associated with each other. The examination ID is identification information assigned to the examination. The patient ID is the patient ID of the patient who is an imaging target. The imaging apparatus is an imaging apparatus used for capturing medical images. The imaging facility is a medical facility where medical images are captured. The examination date and time is the date and time when the examination (imaging) is performed. The image ID is identification information of the medical image relevant to the examination. The interpretation request source user ID is the user ID of the user who makes a request for the interpretation of the examination (medical image). The interpretation request destination user ID is the user ID of the user requested to interpret the examination (medical image). When multiple interpretations are scheduled, a plurality of interpretation request destination user IDs can be designated.

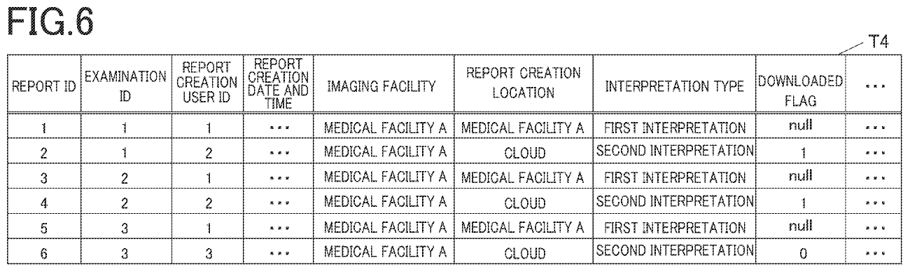

The report management table T4 is a table for managing interpretation reports stored in the data storage region 141. FIG. 6 shows an example of the data configuration of the report management table T4. In the report management table T4, a report ID, an examination ID, a report creation user ID, a report creation date and time, an imaging facility, a report creation location, an interpretation type, a downloaded flag, and the like are stored for each report so as to be associated with each other. The report ID is identification information assigned to the interpretation report. The examination ID is the examination ID of the examination to be interpreted. The report creation user ID is the user ID of the user who creates the interpretation report. The report creation date and time is the date and time when the interpretation report is created. The imaging facility is a medical facility where medical images are captured. The report creation location is the location (the cloud, the medical facility A, the medical facility B, and the like) where the interpretation report is created. The interpretation type is information indicating the type of interpretation, such as first interpretation, second interpretation, or approved interpretation. The downloaded flag is a flag indicating whether or not the interpretation report created in the cloud server 1 has been downloaded to the “imaging facility”. The initial value of the downloaded flag is “0”, and when the interpretation report is downloaded, the downloaded flag becomes “1”. The downloaded flag is set to “null” for interpretation reports created in each medical facility (local servers 3A, 3B, . . . ).

Medical images created in each medical facility, interpretation reports created in each medical facility (local servers 3A, 3B, . . . ), interpretation reports created in the cloud server 1, and the like are stored in the data storage region 141.

The hardware processor 11 acquires a first interpretation report on a first medical image from the local servers 3A, 3B, . . . (hereinafter, may be simply referred to as a local server 3) through the Internet. That is, the hardware processor 11 functions as a first acquirer. The first interpretation report is created within the local network (in-facility networks 6A, 6B, . . . ).

The hardware processor 11 stores the first interpretation report in the storage 14 serving as a first storage.

The hardware processor 11 acquires a second medical image from the local server 3 through the Internet. That is, the hardware processor 11 functions as a second acquirer.

The hardware processor 11 creates a second interpretation report on the second medical image. That is, the hardware processor 11 functions as a creator. The second medical image is assumed to be the same medical image as the first medical image.

The hardware processor 11 creates a second interpretation report by using the first interpretation report. For example, the hardware processor 11 receives an operation, such as correction, addition, and deletion, from the user by using the first interpretation report as a template, thereby creating the second interpretation report. Alternatively, the hardware processor 11 may create a second interpretation report without using the first interpretation report.

The hardware processor 11 stores the second interpretation report in the storage 14 serving as a second storage. Although a case where the first storage that stores the first interpretation report and the second storage that stores the second interpretation report are one storage (storage 14) will be described herein, the first storage and the second storage may be different storages.

The hardware processor 11 controls the communicator 12 to output the second interpretation report to the local server 3 through the Internet. That is, the hardware processor 11 and the communicator 12 function as outputters or transmitters.

The hardware processor 11 receives a report request from the local server 3 through the communicator 12. That is, the hardware processor 11 and the communicator 12 function as inputters or receivers.

The hardware processor 11 outputs the second interpretation report to the local server 3 based on the report request.

The hardware processor 11 controls the client terminals 4A, 4B, 4X, . . . (hereinafter, may be simply referred to as a client terminal 4) connected to each other through the communication network N to display the second interpretation report.

The hardware processor 11 controls the client terminal 4 to display the second interpretation report in a display mode different from the display mode of the first interpretation report. “Display in a different display mode” includes displaying in a different display region and displaying in a different display format.

The hardware processor 11 controls the client terminal 4 to display the second interpretation report so as to be distinguishable from the first interpretation report.

<Configuration of In-Facility System>

Next, each apparatus forming the in-facility system 2A will be described. The local server 3A stores and manages image data of medical images created in the medical facility, interpretation reports created for the medical images, and the like.

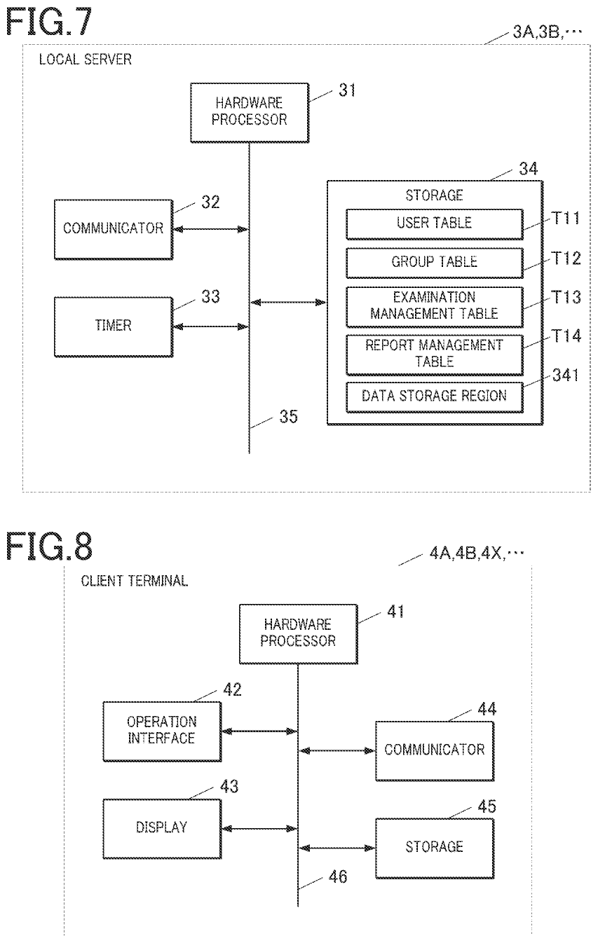

FIG. 7 shows the functional configuration of the local server 3A.

As shown in FIG. 7, the local server 3A is configured to include a hardware processor 31, a communicator 32, a timer 33, a storage 34, and the like, and these are connected to each other by a bus 35.

The hardware processor 31 includes a CPU, a RAM, and the like, and performs overall control of the processing operation of each unit of the local server 3A. Specifically, the CPU reads various processing programs stored in the storage 34, loads the programs to the RAM, and performs various processes in cooperation with the programs.

The communicator 32 includes a network interface and the like, and transmits and receives data to and from an external apparatus connected through the in-facility network 6A or the communication network N.

The timer 33 has a real time clock, and measures the current date and time by using the real time clock and outputs the current date and time to the hardware processor 31.

The storage 34 includes an HDD, an SSD, and the like, and stores various processing programs and parameters, files, and the like necessary for executing the programs. For example, a web server program for realizing a function as a web server that communicates with a web browser installed in an external apparatus in the medical facility A through an HTTP protocol to provide various web screens to the web browser or an application program that operates on a web server and provides an image viewer function, an interpretation report creation function, an interpretation report display function, and the like to the user of an external apparatus through a web browser is stored in the storage 34.

The storage 34 stores a user table T11, a group table T12, an examination management table T13, a report management table T14, and the like. The storage 34 has a data storage region 341.

The user table T11 is similar to the user table T1 shown in FIG. 3, but stores only information of users who belong to the medical facility A.

The group table T12 is similar to the group table T2 shown in FIG. 4, but defines the authority for the local server 3A in the group table T12.

The examination management table T13 is similar to the examination management table T3 shown in FIG. 5, but stores only information of examinations performed in the medical facility A.

The report management table T14 is the same as the report management table T4 shown in FIG. 6 except that there is no downloaded flag. The report management table T14 stores information of interpretation reports created in the medical facility A (local server 3A) and information of interpretation reports downloaded from the cloud server 1 by the local server 3A.

Medical images created in the medical facility A, interpretation reports created in the local server 3A, interpretation reports downloaded from the cloud server 1, and the like are stored in the data storage region 341.

The hardware processor 31 controls the communicator 32 to transmit the second medical image and the first interpretation report created within the local network (in-facility network 6A) to the cloud server 1 through the Internet.

The hardware processor 31 acquires a second interpretation report on the second medical image created in the cloud server 1 through the communicator 32.

The hardware processor 31 causes the storage 34 to store the second interpretation report.

The client terminal 4A is a personal computer (PC) or the like used by medical staff. This is a computer apparatus used when interpreting a medical image to create an interpretation report and when referring to the interpretation report.

FIG. 8 shows the functional configuration of the client terminal 4A.

As shown in FIG. 8, the client terminal 4A is configured to include a hardware processor 41, an operation interface 42, a display 43, a communicator 44, a storage 45, and the like.

The hardware processor 41 includes a CPU, a RAM, and the like, and performs overall control of the processing operation of each unit of the client terminal 4A. Specifically, in response to an operation signal input from the operation interface 42 or an instruction signal received by the communicator 44, the CPU reads various processing programs stored in the storage 45, loads the programs to the RAM, and performs various processes in cooperation with the programs.

The operation interface 42 is configured to include a keyboard having cursor keys, letter and number input keys, various function keys, and the like and a pointing device, such as a mouse, and outputs an operation signal input by a key operation or a mouse operation on the keyboard to the hardware processor 41.

When the client terminal 4A is a tablet terminal, a smartphone, or the like, the operation interface 42 includes operation keys, such as a power key for turning on/off the power, and a touch panel laminated on the display 43, and outputs an operation signal corresponding to each operation key and an operation signal corresponding to the position of a touch operation using the user's finger or the like to the hardware processor 41.

The display 43 is configured to include a monitor, such as a liquid crystal display (LCD), and displays various screens according to instructions of a display signal input from the hardware processor 41. For example, the display 43 displays an interpretation screen (interpretation report creation screen), a report reference screen, and the like.

The communicator 44 includes a network interface and the like, and transmits and receives data to and from an external apparatus connected through the in-facility network 6A or the communication network N.

The storage 45 includes an HDD, an SSD, and the like, and stores various processing programs and parameters, files, and the like necessary for executing the programs. Specifically, the storage 45 stores a web browser program for realizing a web browser and the like.

The imaging apparatus 5A captures an image of a patient's diagnosis target site as a subject and converts the captured image into a digital image to generate image data of the medical image. Examples of the imaging apparatus 5A include computed radiography (CR), computed tomography (CT), magnetic resonance imaging (MR), ultrasonography (US), and endoscope (ES).

The number of imaging apparatuses 5A in the in-facility system 2A is not limited.

Since the in-facility system 2B and the like other than the in-facility system 2A have the same configuration as the in-facility system 2A, the description thereof will be omitted.

Specifically, since the local servers 3B, . . . have the same configuration as the local server 3A, FIG. 7 is referred to and the description thereof will be omitted.

Since the client terminals 4B, . . . in the medical facility have the same configuration as the client terminal 4A, FIG. 8 is referred to and the description thereof will be omitted.

Since the client terminal 4X used inside and outside the medical facility has the same configuration as the client terminal 4A, FIG. 8 is referred to and the description thereof will be omitted.

<Operation in Cloud System>

Next, an operation in the cloud system 100 will be described.

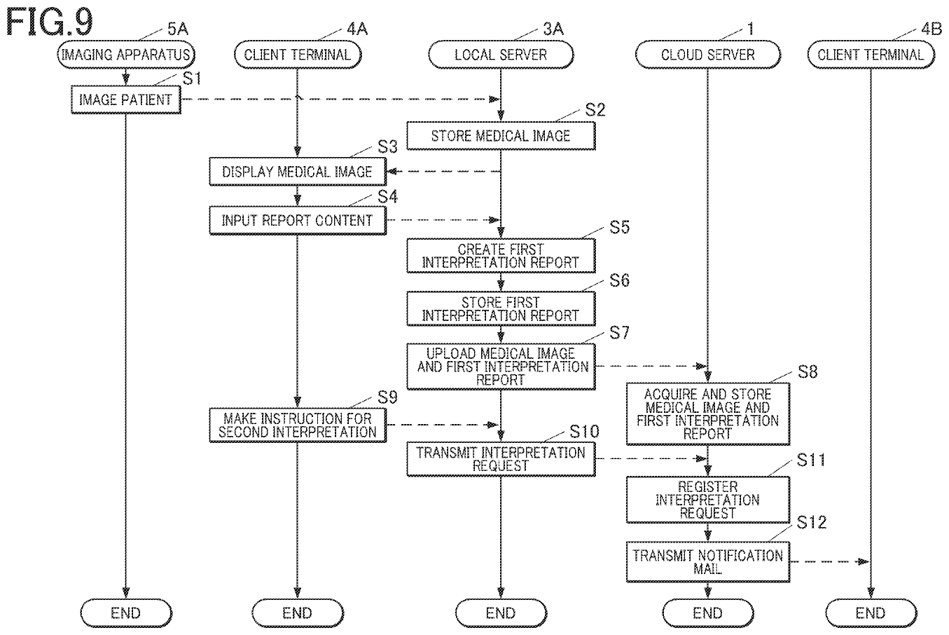

FIG. 9 is a ladder chart showing a first interpretation process performed in the cloud system 100. The first interpretation process is a process including first interpretation of medical images captured in the medical facility A and a request for second interpretation to the medical facility B. The process in the cloud server 1 is realized by software processing in cooperation with the hardware processor 11 and the program stored in the storage 14. The process in the local server 3A is realized by software processing in cooperation with the hardware processor 31 and the program stored in the storage 34. The processes in the client terminals 4A and 4B are realized by software processing in cooperation with the hardware processor 41 and the program stored in the storage 45.

First, the imaging apparatus 5A in the medical facility A captures an image of a patient's site to be examined and generates image data of the medical image (step S1). The imaging apparatus 5A attaches additional information to the medical image by writing the additional information (patient information, examination information, image ID, and the like) in the header of the medical image file in accordance with the DICOM standard. The imaging apparatus 5A transmits the medical image to the local server 3A through the in-facility network 6A.

The patient information is information regarding the patient, and includes a patient ID, a patient name, age, sex, the date of birth, and the like.

The examination information is information regarding the examination, and includes an examination ID, an imaging apparatus, an imaging facility, an examination date and time, an examination site, and the like.

In the local server 3A, upon receiving the medical image through the communicator 32, the hardware processor 31 stores the medical image in the storage 34 (step S2). Specifically, the hardware processor 31 stores the medical image in the data storage region 341 of the storage 34, and stores information (an examination ID, a patient ID, an imaging apparatus, an imaging facility, an examination date and time, an image ID, and the like) of the examination relevant to the medical image in the examination management table T13. The information such as the examination ID, the patient ID, the imaging apparatus, the imaging facility, the examination date and time, and the image ID is acquired from the additional information of the medical image.

Then, when the user (doctor) in the medical facility A logs in to the local server 3A from the client terminal 4A and designates an examination through the operation interface 42, the hardware processor 31 of the local server 3A transmits display data, which is for displaying a medical image corresponding to the designated examination, to the client terminal 4A through the communicator 32. The hardware processor 31 provides the client terminal 4A with an interpretation screen (an image viewer function and an interpretation report creation function).

In the client terminal 4A, a medical image is displayed on the display 43 (step S3). In the group table T12 stored in the storage 34 of the local server 3A, it is assumed that a group to which the user who operates the client terminal 4A belongs has report authority.

Then, when the user in the medical facility A inputs report content through the operation interface 42 of the client terminal 4A (step S4), the hardware processor 31 of the local server 3A creates a first interpretation report based on the input content (step S5), and stores the created first interpretation report in the storage 34 (step S6). Specifically, the hardware processor 31 stores the first interpretation report in the data storage region 341 of the storage 34, and stores information (a report ID, an examination ID, a report creation user ID, a report creation date and time, an imaging facility, a report creation location, an interpretation type, and the like) of the first interpretation report in the report management table T14. The hardware processor 31 assigns a report ID when creating the first interpretation report, and stores the “report ID” in the “report ID” field. The hardware processor 31 stores the “examination ID” of the examination relevant to the medical image to be interpreted in the “examination ID” field. The hardware processor 31 stores the “user ID” of the user who is logging in to the local server 3A from the client terminal 4A in the “report creation user ID” field. The hardware processor 31 stores the “current date and time”, which is acquired from the timer 33 when an instruction to store the first interpretation report is given, in the “report creation date and time” field. The hardware processor 31 stores “medical facility A” in the “imaging facility” field, stores “medical facility A” corresponding to the local server 3A in the “report creation location” field, and stores “first interpretation” in the “interpretation type” field. The first interpretation report is stored, for example, as a file in a portable document format (PDF) or extensible markup language (XML) format.

Then, the hardware processor 31 uploads the interpreted medical image and the first interpretation report to the cloud server 1 through the communicator 32 (step S7).

In the cloud server 1, the hardware processor 11 acquires the medical image and the first interpretation report from the local server 3A through the communicator 12, and stores the acquired medical image and first interpretation report in the storage 14 (step S8). Specifically, the hardware processor 11 stores the medical image and the first interpretation report in the data storage region 141 of the storage 14, stores information (an examination ID, a patient ID, an imaging apparatus, an imaging facility, an examination date and time, an image ID, and the like) of the examination relevant to the medical image in the examination management table T3, and stores information (a report ID, an examination ID, a report creation user ID, a report creation date and time, an imaging facility, a report creation location, an interpretation type, and the like) of the first interpretation report in the report management table T4. The information stored in the examination management table T3 is acquired from additional information of the medical image. The information stored in the report management table T4 is acquired from the local server 3A.

In step S8, the hardware processor 11 of the cloud server 1 acquires the first interpretation report from the local server 3A through the Internet and stores the acquired first interpretation report in the storage 14, and then acquires the second medical image to be the target of the second interpretation report from the local server 3A through the Internet.

Then, when the user in the medical facility A designates an interpretation doctor through the operation interface 42 of the client terminal 4A and makes an instruction for second interpretation (step S9), the hardware processor 31 of the local server 3A transmits an interpretation request to the cloud server 1 through the communicator 32 (step S10). The interpretation request includes the examination ID of the examination to be interpreted, the interpretation request source user ID, and the interpretation request destination user ID. The interpretation request source user ID is the “user ID” of the user who is logging in to the local server 3A from the client terminal 4A. The interpretation request destination user ID is the “user ID” of the designated interpretation doctor.

In the cloud server 1, upon receiving the interpretation request from the local server 3A through the communicator 12, the hardware processor 11 registers the interpretation request in the storage 14 (step S11). Specifically, the hardware processor 11 stores the “interpretation request source user ID” and “interpretation request destination user ID” included in the interpretation request in the record of the examination specified by the “examination ID”, which is included in the interpretation request, in the examination management table T3.

Then, the hardware processor 11 acquires an “email address” corresponding to the “user ID” indicated by the interpretation request destination user ID with reference to the user table T1 in the storage 14, and transmits an interpretation request notification mail to the email address through the communicator 12 (step S12).

A user in the medical facility B is designated as the interpretation request destination. The client terminal 4B in the medical facility B receives the interpretation request notification mail from the cloud server 1 through the communicator 44.

As described above, the first interpretation process ends.

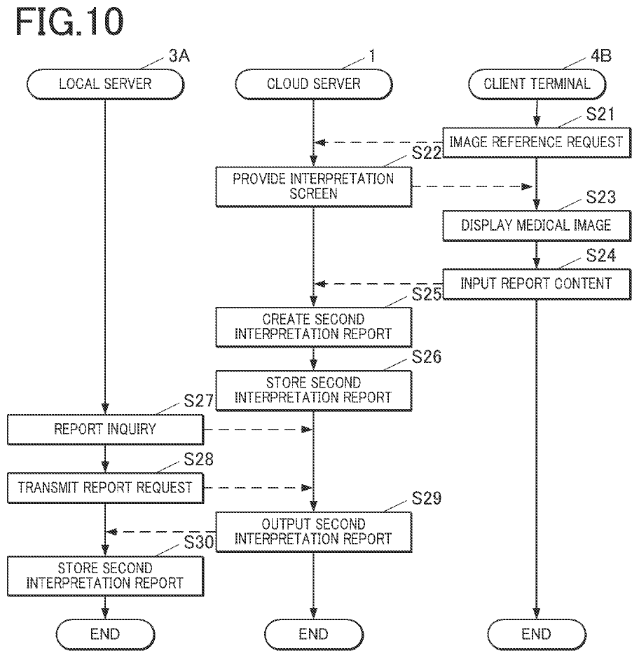

FIG. 10 is a ladder chart showing a second interpretation process performed in the cloud system 100. The second interpretation process is a process including the second interpretation of the medical image and downloading of the second interpretation report, and is performed subsequent to the first interpretation process (see FIG. 9). The process in the cloud server 1 is realized by software processing in cooperation with the hardware processor 11 and the program stored in the storage 14. The process in the local server 3A is realized by software processing in cooperation with the hardware processor 31 and the program stored in the storage 34. The process in the client terminal 4B is realized by software processing in cooperation with the hardware processor 41 and the program stored in the storage 45.

First, when the user (doctor) in the medical facility B requested to perform the second interpretation logs in to the cloud server 1 from the client terminal 4B and designates an examination through the operation interface 42, the hardware processor 41 transmits an image reference request corresponding to the designated examination to the cloud server 1 (step S21) through the communicator 44. In the group table T2 stored in the storage 14 of the cloud server 1, it is assumed that a group to which the user who operates the client terminal 4B belongs has report authority.

Upon receiving the image reference request from the client terminal 4B through the communicator 12, the hardware processor 11 of the cloud server 1 transmits display data for displaying a medical image corresponding to the designated examination to the client terminal 4B. The hardware processor 11 provides the client terminal 4B with an interpretation screen (an image viewer function and an interpretation report creation function) (step S22).

In the client terminal 4B, a medical image is displayed on the display 43 (step S23).

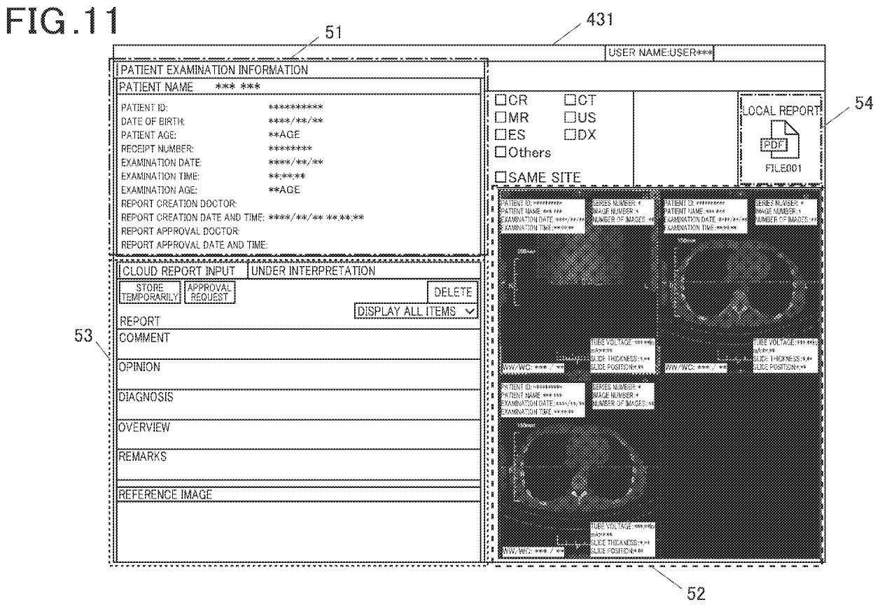

FIG. 11 shows an example of an interpretation screen 431 displayed on the display 43 of the client terminal 4B. The interpretation screen 431 includes a patient examination information region 51, a medical image region 52, a cloud report input region 53, a local report display region 54, and the like.

Patient information and examination information relevant to the examination are displayed in the patient examination information region 51. The patient information and the examination information are acquired from the record corresponding to the designated examination in the examination management table T3 or the additional information of the medical image relevant to the examination by the hardware processor 11 of the cloud server 1.

A medical image relevant to the examination is displayed in the medical image region 52. As for the medical image, the hardware processor 11 of the cloud server 1 acquires an image ID corresponding to the examination ID of the designated examination from the examination management table T3, and acquires a medical image stored in the data storage region 141 based on the image ID.

The cloud report input region 53 is a region for inputting an interpretation report for the medical image displayed in the medical image region 52.

In the local report display region 54, a first interpretation report created in the medical facility A is displayed for the medical image displayed in the medical image region 52. As for the first interpretation report, the hardware processor 11 of the cloud server 1 acquires a report ID corresponding to the examination ID of the designated examination from the report management table T4, and acquires an interpretation report stored in the data storage region 141 based on the report ID. An icon of the PDF file with a file name “FILE001” is shown. When the user clicks on the icon, the content of the first interpretation report (FILE001) is displayed. In the local report display region 54, the fact that the first interpretation report has been created in the medical facility A (local server 3A) may be displayed in a recognizable manner.

Then, when the user in the medical facility B inputs report content through the operation interface 42 of the client terminal 4B (step S24), the hardware processor 11 of the cloud server 1 creates a second interpretation report based on the input content (step S25), and stores the created second interpretation report in the storage 14 (step S26). Specifically, the hardware processor 11 stores the second interpretation report in the data storage region 141 of the storage 14, and stores information (a report ID, an examination ID, a report creation user ID, a report creation date and time, an imaging facility, a report creation location, an interpretation type, and the like) of the second interpretation report in the report management table T4. The hardware processor 11 assigns a report ID when creating the second interpretation report, and stores the “report ID” in the “report ID” field. The hardware processor 11 stores the “examination ID” of the examination relevant to the medical image to be interpreted in the “examination ID” field. The hardware processor 11 stores the “user ID” of the user who is logging in to the cloud server 1 from the client terminal 4B in the “report creation user ID” field. The hardware processor 11 stores the “current date and time”, which is acquired from the timer 13 when an instruction to store the second interpretation report is given, in the “report creation date and time” field. The hardware processor 11 stores “medical facility A” in the “imaging facility” field, stores “cloud” corresponding to the cloud server 1 in the “report creation location” field, and stores “second interpreting” in the “interpretation type” field. When registering a new record in the report management table T4 for the second interpretation report, the hardware processor 11 sets the downloaded flag to “0”. The second interpretation report is stored, for example, as a PDF or XML format file.

The first interpretation report and the second interpretation report may be stored in different file formats so as to be distinguishable and displayed. For example, when the first interpretation report is stored as a PDF format file, the second interpretation report may be stored as an XML format file and displayed. When the first interpretation report is stored as an XML format file, the second interpretation report may be stored as a PDF format file and displayed.

Even when the first interpretation report and the second interpretation report are stored in the same file format, the first interpretation report and the second interpretation report may be stored and displayed so as to be distinguishable by creating the second interpretation report independently of the first interpretation report. For example, the first interpretation report may be stored as a PDF format file and the second interpretation report may be independently stored as another PDF format file, so that the first interpretation report and the second interpretation report may be stored and displayed so as to be distinguishable.

In this manner, the interpretation doctor (user in the medical facility B) accesses the cloud server 1 from the client terminal 4B and interprets a medical image and creates an interpretation report by using the software (application) provided by the cloud server 1.

In the local server 3A of the medical facility A, the hardware processor 31 periodically inquires of the cloud server 1 through the communicator 32 whether or not an interpretation report addressed to the medical facility A has been created (step S27). The interpretation report addressed to the medical facility A is an interpretation report created in response to an interpretation request from the medical facility A.

In the cloud server 1, the hardware processor 11 searches for a record in which the “imaging facility” field is “medical facility A”, the “report creation location” field is “cloud”, and the “downloaded flag” field is “0” with reference to the report management table T4 in the storage 14. If there is a record satisfying the above conditions, the hardware processor 11 notifies the local server 3A through the communicator 12 that the interpretation report addressed to the medical facility A has been created.

The hardware processor 31 of the local server 3A transmits a report request to the cloud server 1 through the communicator 32 in response to the notification from the cloud server 1 (step S28).

Upon receiving the report request from the local server 3A through the communicator 12, the hardware processor 11 of the cloud server 1 reads a second interpretation report (interpretation report addressed to the medical facility A that has not been downloaded) from the data storage region 141 of the storage 14 based on the report request. Then, the hardware processor 11 outputs the second interpretation report to the local server 3A through the communicator 12 (step S29). The hardware processor 11 changes the “downloaded flag” of the record corresponding to the output second interpretation report to “1” in the report management table T4 of the storage 14.

The hardware processor 31 of the local server 3A receives the second interpretation report from the cloud server 1 through the communicator 32, and stores the received second interpretation report in the storage 34 (step S30). Specifically, the hardware processor 31 stores the second interpretation report in the data storage region 341 of the storage 34, and stores information (a report ID, an examination ID, a report creation user ID, a report creation date and time, an imaging facility, a report creation location, an interpretation type, and the like) of the second interpretation report in the report management table T14. The information stored in the report management table T14 is acquired from the cloud server 1.

As described above, the second interpretation process ends.

In the first interpretation process (see FIG. 9) and the second interpretation process (see FIG. 10), the first interpretation report is created in the medical facility A (local server 3A), and the second interpretation report is created in the cloud server 1 by the operation of the doctor in the medical facility B. However, the medical facility where the first interpretation report is created and the interpretation request destination are not limited to the above examples.

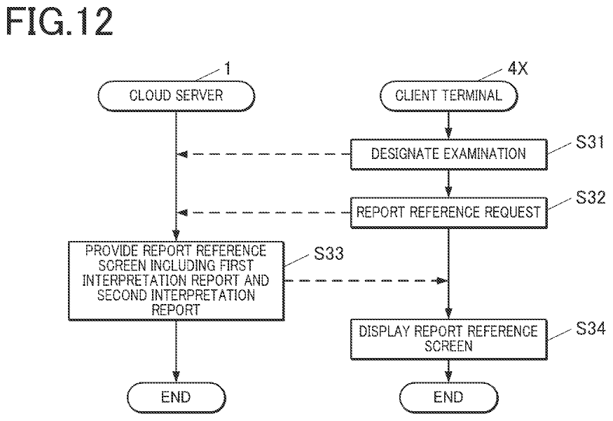

FIG. 12 is a ladder chart showing a report display process performed in the cloud system 100. The report display process is a process for displaying an interpretation report for a medical image. The process in the cloud server 1 is realized by software processing in cooperation with the hardware processor 11 and the program stored in the storage 14. The process in the client terminal 4X is realized by software processing in cooperation with the hardware processor 41 and the program stored in the storage 45.

First, when a user (doctor) who desires to refer to an interpretation report logs in to the cloud server 1 from the client terminal 4X and designates an examination through the operation interface 42 (step S31), the hardware processor 41 transmits a report reference request corresponding to the designated examination to the cloud server 1 through the communicator 44 (step S32). In the group table T2 stored in the storage 14 of the cloud server 1, it is assumed that a group to which the user who operates the client terminal 4X belongs has reference authority.

Upon receiving the report reference request from the client terminal 4X through the communicator 12, the hardware processor 11 of the cloud server 1 transmits display data for displaying an interpretation report corresponding to the designated examination to the client terminal 4X. That is, the hardware processor 11 provides the client terminal 4X with a report reference screen including the first interpretation report and the second interpretation report (step S33). Specifically, the hardware processor 11 acquires a report ID corresponding to the examination ID of the designated examination with reference to the report management table T4 of the storage 14, and acquires the first interpretation report and the second interpretation report from the data storage region 141 of the storage 14 based on the report ID. In the report management table T4, the hardware processor 11 determines a record in which the “report creation location” field is a medical facility such as “medical facility A”, among the records corresponding to the examination ID of the designated examination, to be the “first interpretation report”, and determines a record in which the “report creation location” field is a “cloud” to be the “second interpretation report”. Then, the hardware processor 11 generates display data for a report reference screen including the first interpretation report and the second interpretation report.

In the client terminal 4X, a report reference screen is displayed on the display 43 (step S34).

As described above, the report display process ends.

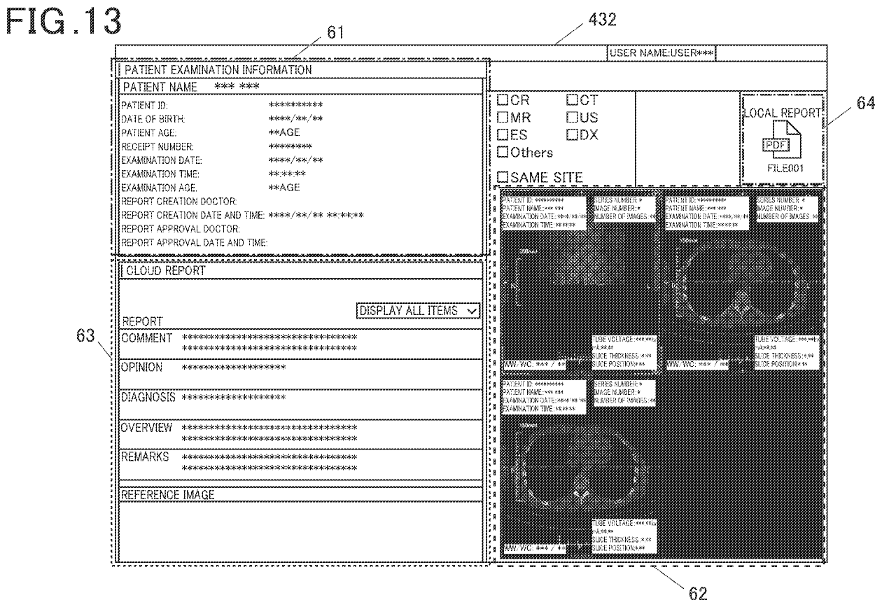

FIG. 13 shows an example of a report reference screen 432 displayed on the display 43 of the client terminal 4X. The report reference screen 432 includes a patient examination information region 61, a medical image region 62, a cloud report display region 63, a local report display region 64, and the like.

The patient examination information region 61 and the medical image region 62 are the same as the patient examination information region 51 and the medical image region 52 of the interpretation screen 431 (see FIG. 11).

In the cloud report display region 63, an interpretation report created in the cloud server 1 is displayed for the medical image displayed in the medical image region 62.

In the local report display region 64, an interpretation report created in each medical facility (local server 3) is displayed for the medical image displayed in the medical image region 62. An icon of the PDF file with a file name “FILE001” is shown.

On the report reference screen 432, the second interpretation report (cloud report) created in the cloud server 1 is displayed in a different display mode from the first interpretation report (local report) created in a server (local server 3) other than the cloud server 1. Specifically, the second interpretation report is displayed in the cloud report display region 63 and the first interpretation report is displayed in the local report display region 64, so that the second interpretation report is displayed in a different region from the first interpretation report. The second interpretation report is displayed in a state in which the content of the report can be referred to, and has a different display mode from the first interpretation report indicated by the icon. The file of the first interpretation report is opened when the user operates the operation interface 42 of the client terminal 4X to click on the icon.

On the report reference screen 432, the second interpretation report created in the cloud server 1 is displayed in a different region and in a different display mode from the first interpretation report created in a server (local server 3) other than the cloud server 1, so that the second interpretation report can be distinguished from the first interpretation report.

When the display 43 of the client terminal 4X has two screen monitors, an interpretation report created in the cloud server 1 may be displayed on one monitor and an interpretation report created in each medical facility (local server 3) may be displayed on the other monitor.

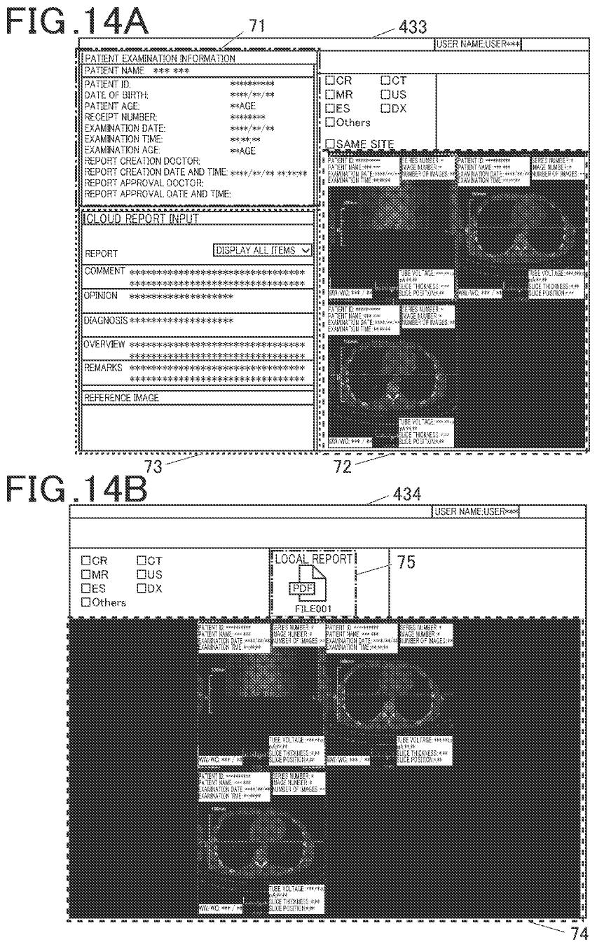

FIG. 14A shows an example of a report reference screen 433 displayed on one monitor of the display 43 of the client terminal 4X.

FIG. 14B shows an example of a report reference screen 434 displayed on the other monitor of the display 43 of the client terminal 4X.

The report reference screen 433 shown in FIG. 14A includes a patient examination information region 71, a medical image region 72, a cloud report display region 73, and the like.

Patient information and examination information relevant to the examination are displayed in the patient examination information region 71.

A medical image relevant to the examination is displayed in the medical image region 72.

In the cloud report display region 73, an interpretation report created in the cloud server 1 is displayed for the medical image displayed in the medical image region 72.

The report reference screen 434 shown in FIG. 14B includes a medical image region 74, a local report display region 75, and the like.

In the medical image region 74, a medical image relevant to the examination is displayed in the same manner as in the medical image region 72.

In the local report display region 75, an interpretation report created in each medical facility (local server 3) is displayed for the medical image displayed in the medical image region 74 (medical image region 72). An icon of the PDF file with a file name “FILE001” is shown. The content of the interpretation report is displayed when the user operates the operation interface 42 of the client terminal 4X to click on the icon.

In the report display process (see FIG. 12), the case where the cloud server 1 causes the client terminal 4X to display the interpretation report is taken as an example. However, the same applies to a case where the cloud server 1 causes other client terminals 4 (client terminals 4A, 4B, . . . ) to display the interpretation report.

The above explanation has been given on the assumption that the group to which the user who operates the client terminal 4X belongs has reference authority in the group table T2 stored in the storage 14 of the cloud server 1. However, assuming that the group to which the user belongs has report authority, the hardware processor 11 can provide the client terminal 4X with an image viewer function and an interpretation report creation function. The provision of the image viewer function and the interpretation report creation function to the client terminal 4X in this case is the same as the case of providing the image viewer function and the interpretation report creation function to the client terminal 4B (see FIG. 10).

As described above, according to the first embodiment, the cloud server 1 can create an interpretation report.

For a medical image uploaded to the cloud server 1, an interpretation report can be created by using the cloud server 1. Therefore, multiple interpretations become easier even in a medical facility where there is only one doctor.

By downloading the interpretation report created in the cloud server 1 from each medical facility (local server 3), even if the medical facility does not belong to a group of affiliated hospitals, interpretation reports created by doctors outside the medical facility can be used.

The cloud server 1 can output the second interpretation report to the local server 3 through the Internet. Specifically, the cloud server 1 can output the second interpretation report to the local server 3 based on the report request from the local server 3.

The second interpretation report created in the cloud server 1 is displayed on the client terminal 4 in a display mode different from the display mode of the first interpretation report created in a server other than the cloud server 1 so as to be distinguishable from the first interpretation report. Therefore, the user can refer to the second interpretation report created in the cloud server 1 in a state in which the second interpretation report created in the cloud server 1 is distinguished from the first interpretation report uploaded from the local server 3.

### Second Embodiment

Next, a second embodiment to which the present invention is applied will be described.

Since a cloud system according to the second embodiment has the same configuration as the cloud system 100 according to the first embodiment, FIGS. 1 to 8 are referred to and the illustration and description of the configuration will be omitted. Hereinafter, the characteristic configuration and processing of the second embodiment will be described.

In the present embodiment, the second interpretation report created in the cloud server 1 is displayed in the same display mode as the first interpretation report created in a server other than the cloud server 1.

The hardware processor 11 of the cloud server 1 controls the client terminal 4 (client terminals 4A, 4B, 4X, . . . ) connected through the communication network N to display the second interpretation report.

The hardware processor 11 controls the client terminal 4 to display the second interpretation report in the same display mode as the display mode of the first interpretation report. “Display in the same display mode” includes displaying in the same display region and displaying in the same display format.

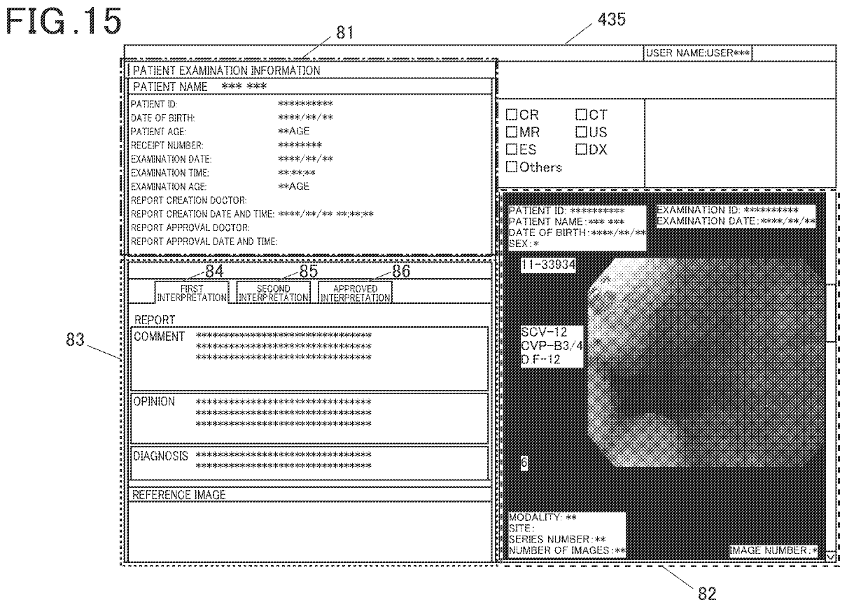

FIG. 15 shows an example of a report reference screen 435 displayed on the display 43 of the client terminal 4. On the report reference screen 435, an interpretation report created in the cloud server 1 and an interpretation report created in each medical facility (local server 3) are displayed in the same display mode. The report reference screen 435 includes a patient examination information region 81, a medical image region 82, a report display region 83, and the like.

Patient information and examination information relevant to the examination are displayed in the patient examination information region 81.

A medical image relevant to the examination is displayed in the medical image region 82.

In the report display region 83, an interpretation report created for the medical image displayed in the medical image region 82 is displayed. A first interpretation tab 84, a second interpretation tab 85, and an approved interpretation tab 86 are provided in the report display region 83, and an interpretation report to be displayed can be switched by selecting each tab.

When displaying the report reference screen 435 on the client terminal 4, the hardware processor 11 of the cloud server 1 extracts a record corresponding to the examination ID of the examination to be displayed with reference to the report management table T4 of the storage 14, and specifies the “interpretation type (first interpretation, second interpretation, approved interpretation, and the like)” of the extracted record. In the extracted record, the “first interpretation report” is when the “report creation location” field is a medical facility such as “medical facility A”, and the “second interpretation report” is when the “report creation location” field is “cloud”.

The hardware processor 11 acquires interpretation reports (the first interpretation report and the second interpretation report) from the data storage region 141 of the storage 14 based on the report ID of the record corresponding to the examination ID of the examination to be displayed, generates display data for displaying the report reference screen 435, and transmits the display data to the client terminal 4 through the communicator 12. The hardware processor 11 displays an interpretation report corresponding to “first interpretation” when the first interpretation tab 84 is selected in the report display region 83 of the report reference screen 435, displays an interpretation report corresponding to “second interpretation” when the second interpretation tab 85 is selected, and displays an interpretation report corresponding to “approved interpretation” when the approved interpretation tab 86 is selected.

On the report reference screen 435, for example, the first interpretation report created in a server (local server 3) other than the cloud server 1 is displayed as an interpretation report corresponding to the “first interpretation”. The second interpretation report created in the cloud server 1 is displayed as an interpretation report corresponding to the “second interpretation”.

In this manner, on the report reference screen 435, the second interpretation report created in the cloud server 1 is displayed in the same display mode as the first interpretation report created in a server (local server 3) other than the cloud server 1. Specifically, the second interpretation report is displayed in the same region and in the same display mode as the first interpretation report.

On the report reference screen 435, it may not be possible to distinguish whether each interpretation report has been created in the cloud server 1 or in a server (local server 3) other than the cloud server 1. However, each interpretation report may be displayed together with the “report creation location” added thereto.

When displaying the second interpretation report in the same display mode as the first interpretation report, the second interpretation report and the first interpretation report may be displayed in the same region, or the second interpretation report and the first interpretation report may be displayed in different display regions while using the same display mode.

Alternatively, the second interpretation report may be created by using the first interpretation report. For example, the second interpretation report may be created by adding or deleting information using the first interpretation report as a template. In this case, the second interpretation report may be created by overwriting the first interpretation report, or the second interpretation report may be created by being stored separately from the first interpretation report. Also in this case, the second interpretation report created in the cloud server 1 can be displayed in the same display mode as the first interpretation report created in a server other than the cloud server 1.

As described above, according to the second embodiment, an interpretation report can be created in the cloud server 1 as in the first embodiment.

Since the second interpretation report created in the cloud server 1 is displayed on the client terminal 4 in the same display mode as the display mode of the first interpretation report created in a server other than the cloud server 1, the user can refer to the second interpretation report created in the cloud server 1 and the first interpretation report uploaded from the local server 3 in the same manner.

### Third Embodiment

Next, a third embodiment to which the present invention is applied will be described. Since a cloud system according to the third embodiment has the same configuration as the cloud system 100 according to the first embodiment, FIGS. 1 to 8 are referred to and the illustration and description of the configuration will be omitted. Hereinafter, the characteristic configuration and processing of the third embodiment will be described.

In the third embodiment, in a predetermined medical facility, an interpretation report stored in the local server 3 is displayed on the client terminal 4.

Taking the medical facility A as an example, the hardware processor 31 of the local server 3A causes the client terminal 4A to display the second interpretation report created in the cloud server 1 in a display mode different from the display mode of the first interpretation report created within the local network (in-facility network 6A).

The hardware processor 31 of the local server 3A causes the client terminal 4A to display the second interpretation report so as to be distinguishable from the first interpretation report.

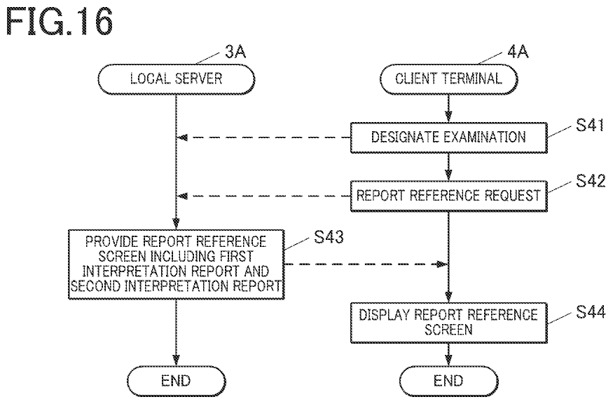

FIG. 16 is a ladder chart showing a second report display process performed by the local server 3A and the client terminal 4A in the medical facility A. The second report display process is a process for displaying an interpretation report for a medical image. The process in the local server 3A is realized by software processing in cooperation with the hardware processor 31 and the program stored in the storage 34. The process in the client terminal 4A is realized by software processing in cooperation with the hardware processor 41 and the program stored in the storage 45.

First, when a user (doctor) who desires to refer to an interpretation report logs in to the local server 3A from the client terminal 4A and designates an examination through the operation interface 42 (step S41), the hardware processor 41 transmits a report reference request corresponding to the designated examination to the local server 3A through the communicator 44 (step S42). In the group table T12 stored in the storage 34 of the local server 3A, it is assumed that a group to which the user who operates the client terminal 4A belongs has reference authority.

Upon receiving the report reference request from the client terminal 4A through the communicator 32, the hardware processor 31 of the local server 3A transmits display data for displaying an interpretation report corresponding to the designated examination to the client terminal 4A. That is, the hardware processor 31 provides the client terminal 4A with a report reference screen including the first interpretation report and the second interpretation report (step S43). Specifically, the hardware processor 31 acquires a report ID corresponding to the examination ID of the designated examination with reference to the report management table T14 of the storage 34, and acquires the first interpretation report and the second interpretation report from the data storage region 341 of the storage 34 based on the report ID. In the report management table T14, the hardware processor 31 determines a record in which the “report creation location” field is “medical facility A”, among the records corresponding to the examination ID of the designated examination, to be the “first interpretation report”, and determines a record in which the “report creation location” field is a “cloud” to be the “second interpretation report”. Then, the hardware processor 31 generates display data for a report reference screen including the first interpretation report and the second interpretation report.

In the client terminal 4A, a report reference screen is displayed on the display 43 (step S44).

As described above, the second report display process ends.

For example, the report reference screen 432 shown in FIG. 13 is displayed on the display 43 of the client terminal 4A. On the report reference screen 432, the second interpretation report (cloud report) created in the cloud server 1 is displayed in a different display mode from the first interpretation report (local report) created within the local network (local server 3A). Specifically, the second interpretation report is displayed in the cloud report display region 63 and the first interpretation report is displayed in the local report display region 64, so that the second interpretation report is displayed in a different region from the first interpretation report. The second interpretation report is displayed in a state in which the content of the report can be referred to, and has a different display mode from the first interpretation report indicated by the icon. The file of the first interpretation report is opened when the user operates the operation interface 42 of the client terminal 4A to click on the icon.

On the report reference screen 432, the second interpretation report created in the cloud server 1 is displayed in a different region and in a different display mode from the first interpretation report created within the local network, so that the second interpretation report can be distinguished from the first interpretation report.

As shown in FIGS. 14A and 14B, the first interpretation report and the second interpretation report may be displayed in two divided screens.

While the local server 3A in the medical facility A has been described above, the same applies to the local server 3 in a medical facility other than the medical facility A.

As described above, according to the third embodiment, the local server 3 causes the client terminal 4 to display the second interpretation report created in the cloud server 1 in a display mode different from the display mode of the first interpretation report created in a server other than the cloud server 1 so that the second interpretation report can be distinguished from the first interpretation report. Therefore, the user can refer to the second interpretation report created in the cloud server 1 in a state in which the second interpretation report created in the cloud server 1 is distinguished from the first interpretation report created within the local network.

Also in the third embodiment, similarly to the second embodiment, the hardware processor 31 of the local server 3 may cause the client terminal 4 to display the second interpretation report in the same display mode as the first interpretation report.

The description in each of the above embodiments is an example of the cloud system according to the present invention, and the present invention is not limited thereto. The detailed configuration and detailed operation of each apparatus forming the system can also be appropriately changed without departing from the spirit of the present invention.

For example, in the first interpretation process shown in FIG. 9, the first interpretation report is created, based on the operation of the doctor in the medical facility A where the medical image is captured, at the timing when the medical image is captured. However, the doctor in the medical facility A may upload only the medical image to the cloud server 1 without creating the first interpretation report.

The timing of the interpretation request is not limited to the above example, and the interpretation request may be made independently at a timing different from when the medical image is uploaded to the cloud server 1.

Whichever of the first interpretation report created for the medical image in the local server 3 and the second interpretation report created for the medical image in the cloud server 1 may be created first.

The first interpretation report created in the local server 3 and the second interpretation report created in the cloud server 1 may be interpretation reports created for different medical images. That is, the second medical image may be a medical image different from the first medical image.

On the interpretation screen or the report reference screen provided to the client terminal 4 by the cloud server 1, patient information and the like may be displayed in an anonymized manner for users who belong to medical facilities other than the medical facility where the patient is imaged.

In each of the embodiments described above, creating an interpretation report in the cloud server 1 or the local server 3 is based on the user's operation input from the client terminal 4. However, the content of the interpretation report does not need to be input by the user. For example, in the cloud server 1 or the local server 3, a medical image may be analyzed by artificial intelligence (AI) to create an interpretation report.

In each of the embodiments described above, in the group tables T2 and T12, the authority (login authority, reference authority, and report authority) is defined for the group to which the user belongs. However, the authority of each user may be set for each examination.

In each of the embodiments described above, the local server 3 manages medical images and interpretation reports. However, in a medical facility, division into a PACS that manages medical images and a report system that manages interpretation reports is possible.

When accessing the cloud server 1 from the client terminal 4A in the medical facility A, if there is an interpretation report created in the medical facility B for a patient in the medical facility A even though the interpretation report is not an interpretation report created in response to the request from the medical facility A, the existence of the interpretation report can be recognized by the user of the client terminal 4A.

When accessing the cloud server 1 from the client terminal 4 and when accessing the local server 3 in the same medical facility as the client terminal 4, the display mode of the interpretation report may be changed based on the approval route or report authority for interpretation. When a patient has medical examinations in a plurality of medical facilities or when a doctor belongs to a plurality of medical facilities, the disclosure range of information may be changed.

The computer-readable medium storing a program for executing each process is not limited to the above example, and a portable recording medium can also be applied. A carrier wave may be applied as a medium for providing program data through a communication line.

While several embodiments of the present invention have been described, the scope of the present invention is not limited to the above-described embodiments, and the scope of the present invention described in the claims and equivalent ranges thereof are included.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. A cloud server that manages a medical image and is connected to a local server through a network, comprising:
a hardware processor that:
acquires a first interpretation report on a first medical image from the local server through an internet connection, and
acquires a second medical image from the local server through the internet connection and creates a second interpretation report on the second medical image;
a first storage that stores the first interpretation report; and
a second storage that stores the second interpretation report.

2. The cloud server according to claim 1, further comprising:
a transmitter that transmits the second interpretation report to the local server through the internet connection.

3. The cloud server according to claim 2, further comprising:
a receiver that receives a report request from the local server, wherein
the transmitter transmits, based on the report request, the second interpretation report to the local server.

4. The cloud server according to claim 1, wherein
the first interpretation report is created within a local network.

5. The cloud server according to claim 1, wherein
the hardware processor creates the second interpretation report by using the first interpretation report.

6. The cloud server according to claim 1, wherein
the hardware processor creates the second interpretation report without using the first interpretation report.

7. The cloud server according to claim 1, wherein
the second medical image is same as the first medical image.

8. The cloud server according to claim 1, wherein
the cloud server is connected to a client terminal through a network, and
the hardware processor:
in a display mode different from a display mode of the first interpretation report, causes the client terminal to display the second interpretation report.

9. The cloud server according to claim 1, wherein
the cloud server is connected to a client terminal through a network, and
the hardware processor:
in a display mode same as a display mode of the first interpretation report, causes the client terminal to display the second interpretation report.

10. The cloud server according to claim 1, wherein
the cloud server is connected to a client terminal through a network, and
the hardware processor:
causes the client terminal to display the second interpretation report to be distinguishable from the first interpretation report.

11. A non-transitory computer-readable medium storing a program to be executed by a computer of a cloud server managing a medical image and included in a cloud system, the cloud server being connected to a local server through a network, the program causing the computer to execute:
acquiring a first interpretation report on a first medical image from the local server through an internet connection;
storing the first interpretation report;
acquiring a second medical image from the local server through the internet connection;
creating a second interpretation report on the second medical image; and
storing the second interpretation report.

12. The computer-readable medium according to claim 11, wherein
the computer executes:
storing the first interpretation report in a storage of the cloud server.

13. The computer-readable medium according to claim 11, wherein
the computer executes:
transmitting the second interpretation report to the local server through the internet connection.

14. The computer-readable medium according to claim 13, wherein
the computer executes:
based on a report request acquired from the local server, transmitting the second interpretation report to the local server.

15. The computer-readable medium according to claim 11, wherein
the first interpretation report is created within a local network.

16. The computer-readable medium according to claim 11, wherein
the computer executes:
creating the second interpretation report by using the first interpretation report.

17. The computer-readable medium according to claim 11, wherein
the computer executes:
creating the second interpretation report without using the first interpretation report.

18. The computer-readable medium according to claim 11, wherein
the second medical image is same as the first medical image.

19. The computer-readable medium according to claim 11, wherein
the computer executes:
in a display mode different from a display mode of the first interpretation report, displaying the second interpretation report on a client terminal.

20. The computer-readable medium according to claim 11, wherein
the computer executes:
in a display mode same as a display mode of the first interpretation report, displaying the second interpretation report on a client terminal.

21. The computer-readable medium according to claim 11, wherein
the computer executes:
displaying the second interpretation report on a client terminal to be distinguishable from the first interpretation report.

22. A non-transitory computer-readable medium storing a program to be executed by a computer of a local server included in a cloud system, the local server being connected through a network to a cloud server that manages a medical image, the program causing the computer to execute:
transmitting a first interpretation report on a first medical image and a second medical image to the cloud server through an internet connection;
acquiring a second interpretation report on the second medical image created in the cloud server; and
storing the second interpretation report.

23. The computer-readable medium according to claim 22, wherein
the computer executes:
in a display mode different from a display mode of the first interpretation report, displaying the second interpretation report on a client terminal.

24. The computer-readable medium according to claim 22, wherein
the computer executes:
in a display mode same as a display mode of the first interpretation report, displaying the second interpretation report on a client terminal.

25. The computer-readable medium according to claim 22, wherein
the computer executes:
displaying the second interpretation report on a client terminal to be distinguishable from the first interpretation report.

26. A cloud system in which a local server and a cloud server that manages a medical image are connected to each other through a network, comprising:
a hardware processor that:
acquires a first interpretation report on a first medical image from the local server through an internet connection,
acquires a second medical image from the local server through the internet connection, and
creates a second interpretation report on the second medical image;
a first storage that stores the first interpretation report; and
a second storage that stores the second interpretation report.

27. The cloud system according to claim 26, further comprising:
a transmitter that transmits the second interpretation report to the local server through the internet connection.

28. The cloud system according to claim 26, wherein
the local server comprises:
a communicator that transmits the first interpretation report and the second medical image to the cloud server through the internet connection, and
the cloud server comprises:
the hardware processor;
the first storage; and
the second storage.

29. A cloud system, comprising:
the cloud server according to claim 1; and
a plurality of local servers connected to the cloud server through a network and including
a first local server and a second local server, wherein
the cloud server is accessible from the first local server and the second local server, and
the first local server and the second local server are inaccessible to each other.
